(19) United States
(12) Patent Application Publication
(10) Pub. No.: US 2019/0261861 A1
(43) Pub. Date: Aug. 29, 2019

(54) METHOD AND APPARATUS FOR EAR IMPRESSION AND ENT IMAGING

(71) Applicant: Caresfream Dental Technology Topco Limited, London (GB)

(72) Inventors: Chuanmao Fan, Rochester, NY (US); Victor C. Wong, Pittsford, NY (US)

(21) Appl. No.: 16/319,804

(22) PCT Filed: Jul. 22, 2016
(86) PCT No.: PCT/US2016/043504

§ 371 Date: Jan. 22, 2019

(51) Int. Cl. A61B 5/00; A61B 5/107; A61B 1/227

(52) U.S. Cl. CPC A61B 5/0066; A61B 5/0073; A61B 5/0088; A61B 1/00082; A61B 5/1079; A61B 1/227; A61B 5/1076

(57) ABSTRACT

An apparatus for imaging a body cavity has a light source that generates short coherence light and a probe housed within a transparent catheter, wherein the probe has a rotatable scanner optic that is disposed to convey light from the light source to and from the body cavity. An actuator apparatus coupled to the probe is configured to perform a helical scan by simultaneously rotating the scanner optic about an axis within the catheter and translating the scanner optic along the axis. A signal detector obtains an interference signal between a first portion of the coherent light scattered from the sample and a second portion of the coherent light reflected from a reference. A processor is programmed with instructions that coordinate actuation of the scanner mirror and acquisition of data from the signal detector and the camera and with instructions for volume image reconstruction.

### FIELD OF THE INVENTION

The disclosure relates generally to methods and apparatus for optical coherence tomography imaging and more particularly to methods and apparatus for ear-nose-throat (ENT) imaging functions.

### BACKGROUND OF THE INVENTION

Optical coherence tomography (OCT) is a non-invasive imaging technique that employs interferometric principles to obtain high resolution, cross-sectional tomographic images that characterize the depth structure of a sample. Particularly suitable for in vivo imaging of human tissue, OCT has shown its usefulness in a range of biomedical research and medical imaging applications, such as in ophthalmology, dermatology, oncology, and other fields, as well as in ear-nose-throat (ENT) and dental imaging.

OCT has been described as a type of “optical ultrasound”, imaging reflected energy from within living tissue to obtain cross-sectional data. In an OCT imaging system, light from a wide-bandwidth source, such as a super luminescent diode (SLD) or other light source, is directed along two different optical paths: a reference arm of known length and a sample arm that illuminates the tissue or other subject under study. Reflected and back-scattered light from the reference and sample arms is then recombined in the OCT apparatus and interference effects are used to determine characteristics of the surface and near-surface underlying structure of the sample. Interference data can be acquired by rapidly scanning the sample illumination across the sample. At each of several thousand points, OCT apparatus obtains an interference profile which can be used to reconstruct an A-scan with an axial depth into the material that is a factor of light source coherence. For most tissue imaging applications, OCT uses broadband illumination sources and can provide image content at depths of a few millimeters (mm).

Initial OCT apparatus employed a time-domain (TD-OCT) architecture in which depth scanning is achieved by rapidly changing the length of the reference arm using some type of mechanical mechanism, such as a piezoelectric actuator, for example. TD-OCT methods use point-by-point scanning, requiring that the illumination probe be moved or scanned from one position to the next during the imaging session. More recent OCT apparatus use a Fourier-domain architecture (FD-OCT) that discriminates reflections from different depths according to the optical frequencies of the signals they generate. FD-OCT methods simplify or eliminate axial scan requirements by collecting information from multiple depths simultaneously and offer improved acquisition rate and signal-to-noise ratio (SNR). There are two implementations of Fourier-domain OCT: spectral domain OCT (SD-OCT) and swept-source OCT (SS-OCT).

SD-OCT imaging can be accomplished by illuminating the sample with a broadband illumination source and dispersing the reflected and scattered light with a spectrometer onto an array detector, such as a CCD (charge-coupled device) detector, for example. SS-OCT imaging illuminates the sample with a rapid wavelength-tuned laser and collects light reflected during a wavelength sweep using only a single photodetector or balanced photodetector. With both SD-OCT and SS-OCT, a profile of scattered light reflected from different depths is obtained by operating on the recorded interference signals using Fourier transforms, such as Fast-Fourier transforms (FFT), well known to those skilled in the signal analysis arts.

Because of their potential to achieve higher performance at lower cost, FD-OCT systems based on swept-frequency laser sources have attracted significant attention for medical applications that require subsurface imaging in highly scattering tissues.

One of the challenges to SS-OCT is providing a suitable light source that can generate the needed sequence of wavelengths in rapid succession. To meet this need, swept-source OCT systems conventionally employ a high-speed wavelength sweeping laser that is equipped with an intracavity monochrometer or uses some type of external cavity narrowband wavelength scanning filter for tuning laser output. Examples of external devices that have been used for this purpose include a tunable Fabry-Perot filter whose cavity length is adjusted to provide a linear change of longitudinal mode, and a polygon scanner filter that selectively reflects dispersive wavelength light. Fourier domain mode locking is a recently reported technique that has been used to generate a sweeping frequency, generally most useful for OCT imaging using broadband near infrared (BNIR) wavelengths.

References for providing a tunable laser include the following:
S. R. Chinn, E. A. Swanson, and J. G. Fujimoto, “Optical coherence tomography using a frequency-tunable optical source,” Opt. Lett. 22, 340-342 (1997).
B. Golubovic, B. E. Bouma, G. J. Tearney, and J. G. Fujimoto, “Optical frequency-domain reflectometry using rapid wavelength tuning of a Cr4+:forsterite laser,” Opt. Lett. 22, 1704-1706 (1997).
S. H. Yun, C. Boudoux, G. J. Tearney, and B. E. Bouma, “High-speed wavelength-swept semiconductor laser with a polygon-scanner-based wavelength filter,” Opt. Lett. 28, 1981-1983 (2003).
Woojin Shin, Boan-Ahn Yu, Yeung Lak Lee, Tae Jun Yu, Tae Joong Eom, Young-Chul Noh, Jongmin Lee, and Do-Kyeong Ko, “Tunable Q-switched erbium-doped fiber laser based on digital micromirror array,” Opt. Express 14, 5356-5364 (2006).
Xiao Chen, Bin-bin Yan, Fei-jun Song, Yi-quan Wang, Feng Xiao, and Kamal Alameh, “Diffraction of digital micro-mirror device gratings and its effect on properties of tunable fiber lasers,” Appl. Opt. 51, 7214-7220 (2012).

Reference is also made to the following:
Huang, D; Swanson, E A; Lin, C P; Schuman, J S; Stinson, W G; Chang, W; Hee, M R; Flotte, T et al. (1991). “Optical coherence tomography”. Science 254 (5035): 1178-81. Bibcode: 1991Sci . . . 254.1178H. doi:10.1126/science.1957169.PMID 1957169.
U.S. Pat. No. 7,355,721 B2, “Optical coherence tomography imaging” to Quadling et al.
U.S. Pat. No. 8,345,261 B2, “Optical coherence tomography imaging” to Quadling et al.
U.S. Pat. Nos. 8,928,888 B2 and 8,345,257 B2 , “Swept source optical coherence tomography (OCT) method and system”, both to Bonnema et al.
U. S. Patent Application No. US20130330686A1 entitled “ Dental optical measuring device and dental optical measuring/diagnosing tool” by Kaji et al.
Hung, K.-W.; Siu, W.-C., “Fast image interpolation using the bilateral filter,” in Image Processing, IET, vol. 6, no. 7, pp. 877-890, October 2012. doi: 10.1049/iet-ipr.2011.0050.
Evgeniy Lebed, Paul J. Mackenzie, Marinko V. Sarunic, and Faisal M. Beg, “Rapid Volumetric OCT Image Acquisition Using Compressive Sampling,” Opt. Express 18, 21003-21012 (2010).
Xuan Liu and Jin U. Kang, “Compressive SD-OCT: the application of compressed sensing in spectral domain optical coherence tomography,” Opt. Express 18, 22010-22019 (2010).
U.S. Pat. No. 8,135,453, Method and apparatus for ear canal surface modeling using optical coherence tomography imaging, to Slabaugh et al.
U.S. Patent Application Publn. No. 2014/0276022, Micropositioner and head holder for cochlear endoscopy, to Oghalai et al.

Accurate 3D measurement of the outer ear shape is useful to help provide a suitable fit for customized hearing treatment devices. In conventional practice, a silicone impression is obtained using methods that have proved to be cumbersome and often less than accurate, often with measurement errors of as much as a millimeter. Optical methods that have been proposed include methods using optical triangulation and confocal imaging. These methods, however, are hampered by the need to operate within tightly confined space.

Thus, it can be appreciated that there is a need for improved scanning apparatus and methods for ENT imaging using OCT.

### SUMMARY OF THE INVENTION

It is an object of the present disclosure to advance the art of diagnostic imaging and to address the need for more accurate imaging of the ear canal and related structures, as well as other imaging for ENT (ear, nose, and throat) applications. Exemplary method and/or apparatus embodiments according to the application can employ OCT scanning using an endoscopic probe for obtaining more accurate spatial characterization of inner surfaces of the anatomy.

These objects are given only by way of illustrative example, and such objects may be exemplary of one or more embodiments of the invention. Other desirable objectives and advantages inherently achieved by the disclosed methods may occur or become apparent to those skilled in the art. The invention is defined by the appended claims.

According to an aspect of the application, there is provided an apparatus for imaging a body cavity that can include a light source that generates short coherence light, a probe housed within a catheter, wherein the probe has a rotatable scanner optic that is disposed to convey the coherent light outwards from the light source to the body cavity and to obtain scattered light from the body cavity, an actuator apparatus coupled to the probe and configured to perform a helical scan by simultaneously rotating the scanner optic about an axis within the catheter and translating the scanner optic along the axis, a signal detector that obtains an interference signal between a first portion of the coherent light scattered from the body cavity and a second portion of the coherent light reflected from a reference, and a processor that is programmed with instructions that coordinate helical scan timing of the scanner optic and acquisition of data from the signal detector and further programmed with instructions for volume image reconstruction.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other. Some exaggeration may be necessary in order to emphasize basic structural relationships or principles of operation. Some conventional components that would be needed for implementation of the described embodiments, such as support components used for providing power, for packaging, and for mounting and protecting system optics, for example, are not shown in the drawings in order to simplify description.

FIG. 1 is a schematic diagram that shows a programmable filter according to an exemplary embodiment of the application.

FIG. 2A is a simplified schematic diagram that shows how the programmable filter provides light of a selected wavelength band.

FIG. 2B is an enlarged view of a portion of the micro-mirror array of the programmable filter.

FIG. 3 is a plan view that shows the arrangement of micro-mirrors in the array.

FIG. 4 is a schematic diagram that shows a programmable filter using a prism as its dispersion optic, according to an alternate exemplary embodiment of the application

FIG. 5 is a schematic diagram showing a programmable filter that performs wavelength-to-wavenumber transformation, according to an alternate exemplary embodiment of the application.

FIG. 6A is a schematic diagram showing a swept-source OCT (SS-OCT) apparatus using a programmable filter according to an embodiment of the present disclosure that uses a Mach-Zehnder interferometer.

FIG. 6B is a schematic diagram showing a swept-source OCT (SS-OCT) apparatus using a programmable filter according to an embodiment of the present disclosure that uses a Michelson interferometer.

FIG. 7 is a schematic diagram that shows a tunable laser using a programmable filter according to an exemplary embodiment of the application.

FIG. 8 is a schematic diagram that shows use of a programmable filter for selecting a wavelength band from a broadband light source.

FIG. 9 shows a helical OCT scan pattern for scanning a body cavity according to exemplary embodiments.

FIG. 10 is a schematic diagram that shows components of an OCT imaging system for ENT imaging according to one exemplary embodiment.

FIG. 11 is a simplified schematic diagram showing portions of an acquisition and processing sequence for OCT imaging of the ear canal or similar volume using the scan data obtained according to an exemplary embodiment of the application.

FIG. 12 is a schematic diagram that shows an exemplary imaging system embodiment for ear canal characterization.

FIG. 13 is a schematic diagram that shows an exemplary imaging system embodiment for ear canal characterization using swept-source spectral domain optical coherence tomography.

FIG. 14 is an exemplary timing diagram that shows synchronization of probe rotational movement to image acquisition timing.

FIG. 15 is an exemplary timing diagram that shows relative timing and synchronization of probe motor rotation angle to stepping motor actuation.

FIG. 16 shows an exemplary ENT probe embodiment according to an exemplary embodiment of the application.

FIG. 17 is a schematic diagram showing components of an ENT probe embodiment according to another exemplary embodiment of the application.

FIG. 18 is a schematic diagram showing components of an ENT probe embodiment according to yet another exemplary embodiment of the application with stabilizing devices.

FIG. 19 is a cross-sectional view that shows positioning an exemplary ENT probe embodiment that includes a resuscitation tube.

### DESCRIPTION OF EXEMPLARY EMBODIMENTS

The following is a description of exemplary method and/or apparatus embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the term “optics” is used generally to refer to lenses and other refractive, diffractive, and reflective components or apertures used for shaping and orienting a light beam. An individual component of this type is termed an optic.

In the context of the present disclosure, the terms “viewer”, “operator”, and “user” are considered to be equivalent and refer to the viewing practitioner, technician, or other person who may operate a camera or scanner and may also view and manipulate an image, such as a dental image, on a display monitor. An “operator instruction” or “viewer instruction” is obtained from explicit commands entered by the viewer, such as by clicking a button on the camera or scanner or by using a computer mouse or by touch screen or keyboard entry.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “camera” relates to a device that is enabled to acquire a reflectance, 2-D digital image from reflected visible or NIR (near-infrared) light, such as structured light that is reflected from the surface of teeth and supporting structures.

The general term “scanner” relates to an optical sensor that projects a scanned light beam of broadband near-IR (BNIR) illumination that is directed to the tooth surface through a sample arm and acquired, as scattered light returned in the sample arm, for detecting interference with light from a reference arm used in OCT imaging of a surface. The term “raster scanner” relates to the combination of hardware components that scan light toward a sample, as described in more detail subsequently. Scanner optics can include a rotating mirror or prism, for example.

The term “subject” refers to the tooth or other portion of a patient that is being imaged and, in optical terms, can be considered equivalent to the “object” of the corresponding imaging system.

In the context of the present disclosure, the phrase “broadband light emitter” refers to an illumination or light source that emits a continuous spectrum output over a range of wavelengths at any given point of time. Short coherence light is equivalently termed low coherence light in the context of the present disclosure and has the meaning and overall small coherence range conventionally understood by those skilled in the optical interferometry arts. Short-coherence or low-coherence, broadband illumination sources can include, for example, super luminescent diodes, short-pulse lasers, many types of white-light sources, and supercontinuum light sources. Most low coherence length sources of these types have a coherence length on the order of tens of microns or less. In general, very low temporal coherence (but suitable spatial coherence) is required for coherence tomography, where images are created using interferometry techniques; high spatial resolution requires low temporal coherence.

As is well-known to those skilled in the OCT imaging arts, the axial resolution is inversely proportional to the coherence length of the light source. Thus, within the range available using OCT imaging, the shorter the coherence length, the higher the axial resolution.

Certain exemplary method and/or apparatus embodiments of the application can utilize any of a number of types of OCT scanning methods, including time-domain or spectral or frequency-domain OCT. Because the speed advantage is of particular interest, the description that follows is primarily directed to embodiments that employ swept-source OCT, a type of frequency-domain OCT that is generally advantageous for faster speed and overall scanning throughput. Exemplary method embodiments of the application can also be used where a spectrometer is used for sensing in the OCT system.

According to an exemplary embodiment of the application, there is provided a programmable light source that can provide variable wavelength illumination that has advantages for improved OCT scanning methods as described herein. The programmable light source can be used as a swept-source for scanned SS-OCT and other applications that benefit from a controllably changeable spectral pattern.

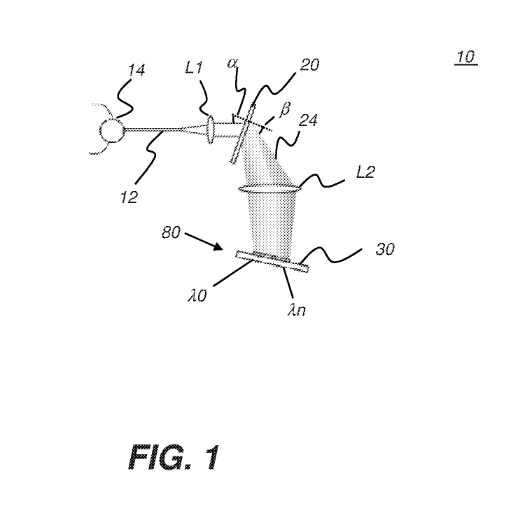

Referring to FIG. 1, there is shown a programmable filter 10 that is used for generating a desired pattern and sequence of wavelengths (λ0 . . . λn) from a low-coherence, broadband light source. Broadband light from a fiber laser or other source is directed, through a circulator 14 through an optical fiber or other waveguide 12 to a collimator lens L1 that directs the collimated light to a light dispersion optic 20, such as a diffraction grating. Light dispersion optic 20 forms a spectrally dispersed output beam 24, directed toward a focusing lens L2. Lens L2 focuses the dispersed light onto a spatial light modulator 80, such as a micro-mirror array 30. The micro-mirror array can be a linear array of reflective devices or a linear portion of a Digital Light Processor (DLP) from Texas Instruments, Dallas, Tex. One or more individual reflectors in array 30 is actuated to reflect light of corresponding wavelengths back through the optical path. This reflected light is the output of programmable filter 10 and can be used in applications such as optical coherence tomography (OCT) as described subsequently. Rapid actuation of each successive reflector in array 30 allows sampling of numerous small spectral portions of a spectrally dispersed output beam, such as that provided in FIG. 1. For example, where the spatial light modulator 80 is a micro-mirror array 30 that has 2048 micro-mirror elements in a single row, where the spectral range from one side of the array 30 to the other is 35 nm, each individual micro-mirror can reflect a wavelength band that is approximately 0.017 nm wide. One typical swept source sequence advances from lower to higher wavelengths by actuating a single spatial light modulator 80 pixel (reflective element) at a time, along the line formed by the spectrally dispersed output beam. Other swept source sequences are possible, as described subsequently.

The micro-mirror array 30 described herein and shown in FIGS. 1-3 and following is one type of possible spatial light modulator 80 that can be used as part of a programmable light source. The spatial light modulator 80 that is employed is a reflective device of some type, with discretely addressable elements that effectively provide the “pixels” of the device.

Programmable filter 10 resembles aspects of a spectrometer in its overall arrangement of components and in its light distribution. Incident broadband BNIR light is dispersed by light dispersion optic 20 in order to spatially separate the spectral components of the light. The micro-mirror array 30 or other type of spatial light modulator 80, as described in more detail subsequently, is disposed to reflect a selected wavelength band or bands of this light back through programmable filter 10 so that the selected wavelength band can be used elsewhere in the optical system, such as for use in an interferometry measurement device or for tuning a laser.

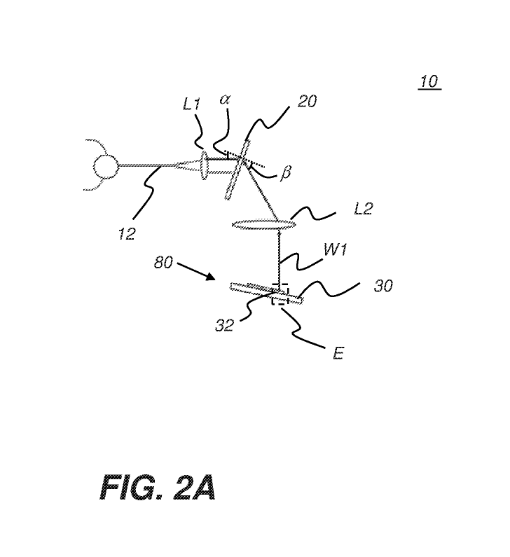

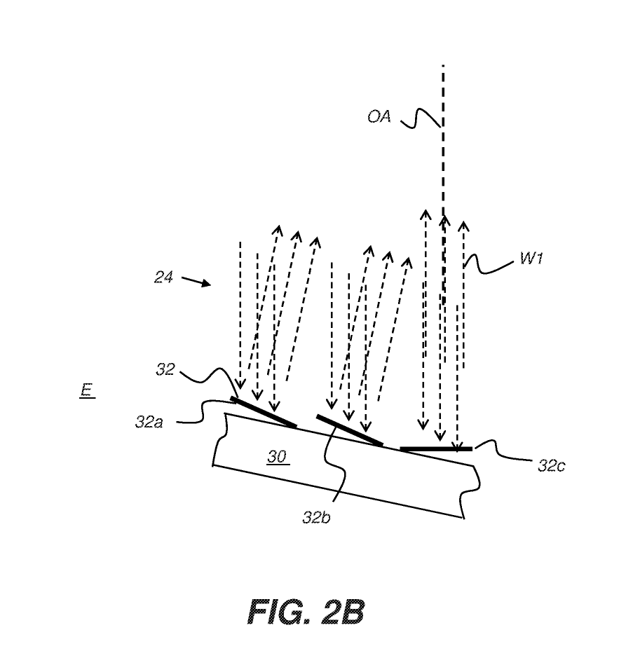

The simplified schematic of FIG. 2A and enlargement of FIG. 2B show how programmable filter 10 operates to provide light of a selected wavelength band W1. FIG. 2B, which schematically shows a greatly enlarged area E of micro-mirror array 30, shows the behavior of three mirrors 32a, 32b, and 32c with respect to incident light of beam 24. Each mirror 32 element of micro-mirror array 30 can have either of two states: deactuated, tilted at one angle, as shown at mirrors 32a and 32b; or actuated, tilted at an alternate angle as shown at mirror 32c. For DLP devices, the tilt angles for deactuated/actuated states of the micro-mirrors are +12 and −12 degrees from the substrate surface. Thus, in order to direct light back along optical axis OA through lens L2 and through the other components of programmable filter 10, micro-mirror array 30 is itself tilted at +12 degrees relative to the optical axis OA, as shown in FIG. 2B.

In the programmable filter 10 of FIG. 1, light dispersion optic 20 can be a diffraction grating of some type, including a holographic diffraction grating, for example. The grating dispersion equation is:

mλ=d(sin α+sin β) (eq. 1)

wherein:
λ is the optical wavelength;
d is the grating pitch;
α is the incident angle (see FIGS. 1, 2A), relative to a normal to the incident surface of optic 20;
β is the angle of diffracted light, relative to a normal to the exit surface of optic 20;
m is the diffraction order, generally m=1 with relation to embodiments of the application.

The FWHM (full-width half-maximum) bandwidth is determined by the spectral resolution of the grating δλ_{g} and wavelength range on a pixel or micro-mirror 32 of the DLP device δλ_{DLP}, which are given as:

δλ_{g}=λc d cos α/D (eq. 2)

and

δλ_{DLP}=dp cos β/f. (eq. 3)

wherein:

D is the 1/e² width of the incident Gaussian beam collimated by lens L1;

λc is the central wavelength;

d is the grating pitch;

p is the DLP pixel pitch, for each micro-mirror;

f is the focus length of focus lens L2.

The final FWHM bandwidth δλ is the maximum of (δλ_{g}, δλ_{DLP}). Bandwidth δλ defines the finest tunable wavelength range. For a suitable configuration for OCT imaging, the following relationship holds:

δλ_{g}≤δλ_{DLP}.

In order to use the DLP to reflect the light back to the waveguide 12 fiber, the spectrally dispersed spectrum is focused on the DLP surface, aligned with the hinge axis of each micro-mirror 32. The DLP reference flat surface also tilts 12 degrees so that when a particular micro-mirror 32 is in an “on” state, the light is directly reflected back to the optical waveguide 12. When the micro-mirror is in an “on” state, the corresponding focused portion of the spectrum, with bandwidth corresponding to the spatial distribution of light incident on that micro-mirror, is reflected back to the waveguide 12 fiber along the same path of incident light, but traveling in the opposite direction. Circulator 14 in the fiber path guides the light of the selected spectrum to a third fiber as output. It can be readily appreciated that other types of spatial light modulator 80 may not require orientation at an oblique angle relative to the incident light beam, as was shown in the example of FIG. 2B.

The 1/e² Gaussian beam intensity diameter focused on a single DLP pixel is as follows:

w=4λ f/(πD cos β/cos α) (eq. 4)

Preferably, the following holds: w≤p. This sets the beam diameter w at less than the pixel pitch p. The maximum tuning range is determined by:

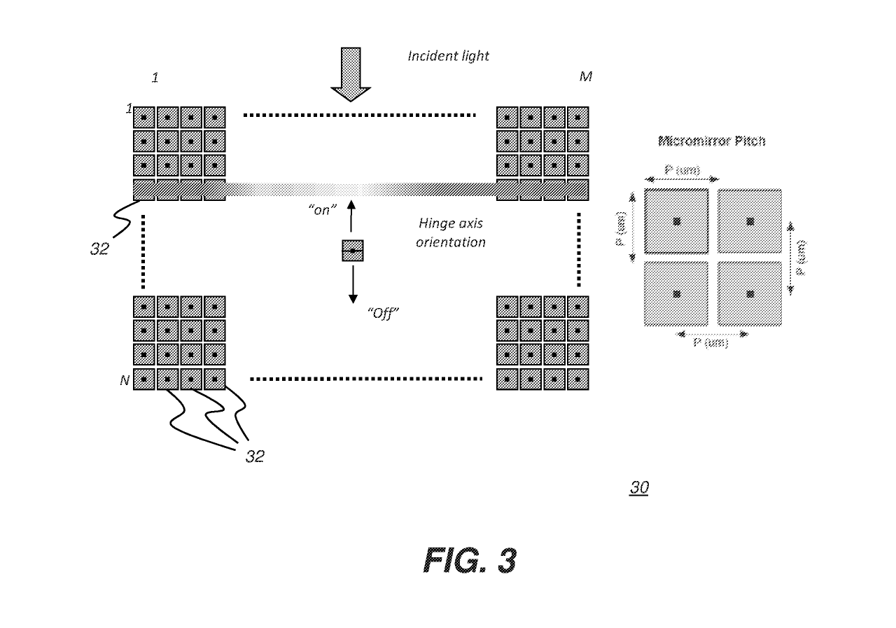

M×δλ_{DLP},

wherein M is the number of DLP micro-mirrors in the horizontal direction, as represented in FIG. 3. As FIG. 3 shows, the array of micro-mirrors for micro-mirror array 30 has M columns and N rows. Only a single row of the DLP micro-mirror array is needed for use with programmable filter 10; the other rows above and below this single row may or may not be used.

The wavelength in terms of DLP pixels (micro-mirrors) can be described by the following grating equation:

$${λ}_{i} = d ( sin α + sin ( {tan}^{- 1} [ \frac{p}{f} ( \frac{N}{2} - i - 1 ) ] + β ) ) \qquad ( eq . 5 )$$

Wherein i is an index for the DLP column, corresponding to the particular wavelength, in the range between 0 and (M-1).

From the above equation (5), the center wavelength corresponding to each mirror in the row can be determined.

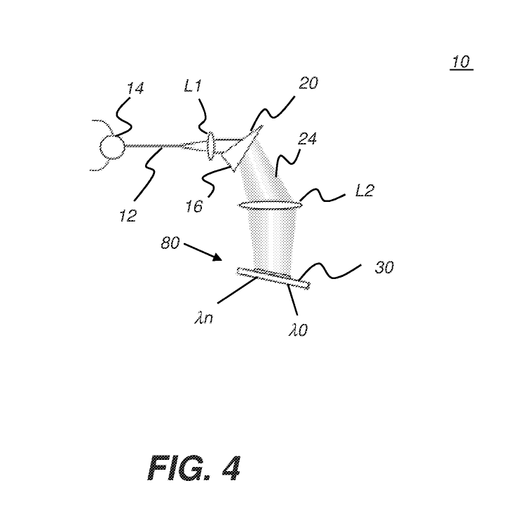

FIG. 4 shows programmable filter 10 in an alternate embodiment, with a prism 16 as light dispersion optic 20. The prism 16 disperses the light wavelengths (λn . . . λ0) in the opposite order from the grating shown in FIG. 1. Longer wavelengths (red) are dispersed at a higher angle, shorter wavelengths (blue) at lower angles.

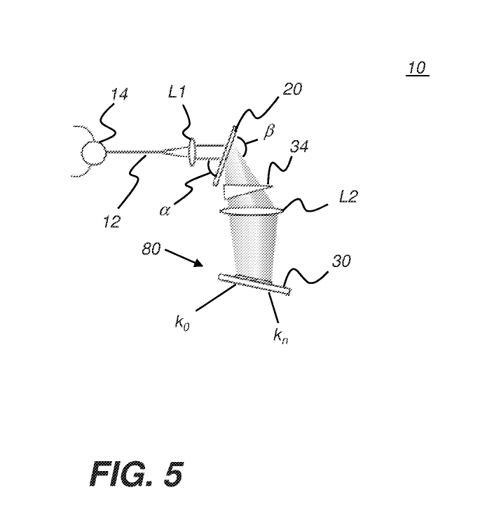

Conventional light dispersion optics distribute the dispersed light so that its constituent wavelengths have a linear distribution. That is, the wavelengths are evenly spaced apart along the line of dispersed light. However, for Fourier domain OCT processing, conversion of wavelength data to frequency data is needed. Wavelength data (λ in units of nm) must thus be converted to wave-number data (k=λ⁻¹), proportional to frequency. In conventional practice, an interpolation step is used to achieve this transformation, prior to Fourier transform calculations. The interpolation step requires processing resources and time. However, it would be most advantageous to be able to select wave-number k values directly from the programmable filter. The schematic diagram of FIG. 5 shows one method for optical conversion of wavelength (λ₀ . . . λ_{N}) data to wave-number (k₀ . . . k_{N}) data using an intermediate prism 34. Methods for specifying prism angles and materials parameters for wavelength-to-wavenumber conversion are given, for example, in an article by Hu and Rollins entitled “Fourier domain optical coherence tomography with a linear-in-wavenumber spectrometer” in OPTICS LETTERS, Dec. 15, 2007, vol. 32 no. 24, pp. 3525-3527.

Programmable filter 10 is capable of providing selected light wavelengths from a broadband light source in a sequence that is appropriately timed for functions such as OCT imaging using a tuned laser. Because it offers a programmable sequence, the programmable filter 10 can perform a forward spectral sweep from lower to higher wavelengths as well as a backward sweep in the opposite direction, from higher to lower wavelengths. A triangular sweep pattern, generation of a “comb” of wavelengths, or arbitrary wavelength pattern can also be provided.

For OCT imaging in particular, various programmable sweep paradigms can be useful to extract moving objects in imaging, to improve sensitivity fall-off over depth, etc. The OCT signal sensitivity decreases with increasing depth into the sample, with depth considered to extend in the z-axis direction. Employing a comb of discrete wavelengths, for example, can increase OCT sensitivity. This is described in an article by Bajraszewski et al. entitled “Improved spectral optical coherence tomography using optical frequency comb” in Optics Express, Vol. 16 No. 6, March 2008, pp. 4163-4176.

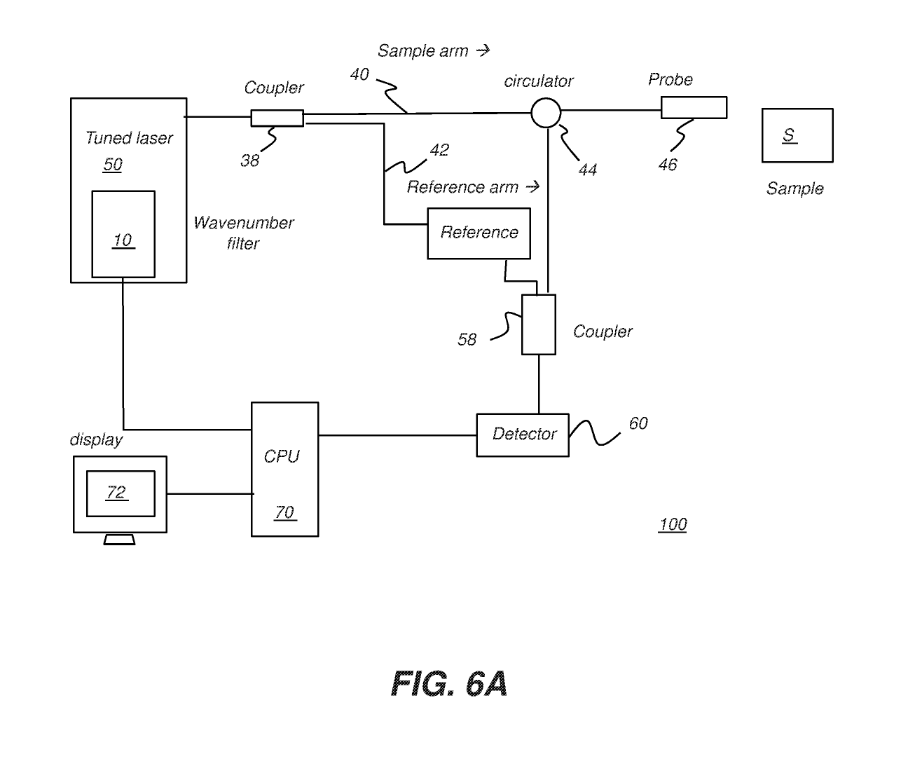

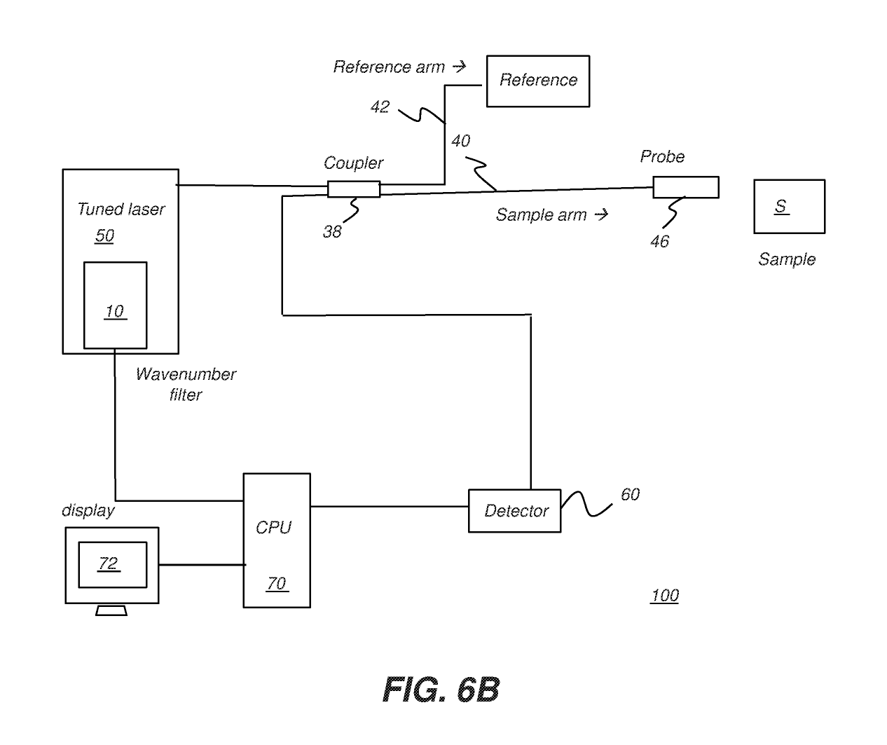

The simplified schematic diagrams of FIGS. 6A and 6B each show a swept-source OCT (SS-OCT) apparatus 100 using programmable filter 10 according to an embodiment of the application. In each case, programmable filter 10 is used as part of a tuned laser 50 that provides an illumination source. For intraoral OCT, for example, laser 50 can be tunable over a range of frequencies (wave-numbers k) corresponding to wavelengths between about 400 and 1600 nm. According to an embodiment of the application, a tunable range of 35 nm bandwidth, centered about 830 nm, is used for intraoral OCT.

In the FIG. 6A embodiment, a Mach-Zehnder interferometer system for OCT scanning is shown. FIG. 6B shows components for a Michelson interferometer system. For these embodiments, programmable filter 10 provides part of the laser cavity to generate tuned laser 50 output. The variable laser 50 output goes through a coupler 38 and to a sample arm 40 and a reference arm 42. In FIG. 6A, the sample arm 40 signal goes through a circulator 44 and to a probe 46 for measurement of a sample S. The sampled signal is directed back through circulator 44 (FIG. 6A) and to a detector 60 through a coupler 58. In FIG. 6B, the signal goes directly to sample arm 40 and reference arm 42; the sampled signal is directed back through coupler 38 and to detector 60. The detector 60 may use a pair of balanced photodetectors configured to cancel common mode noise. A control logic processor (central processing unit CPU) 70 is in signal communication with tuned laser 50 and its programmable filter 10 and with detector 60 and obtains and processes the output from detector 60. CPU 70 is also in signal communication with a display 72 for command entry and OCT results display.

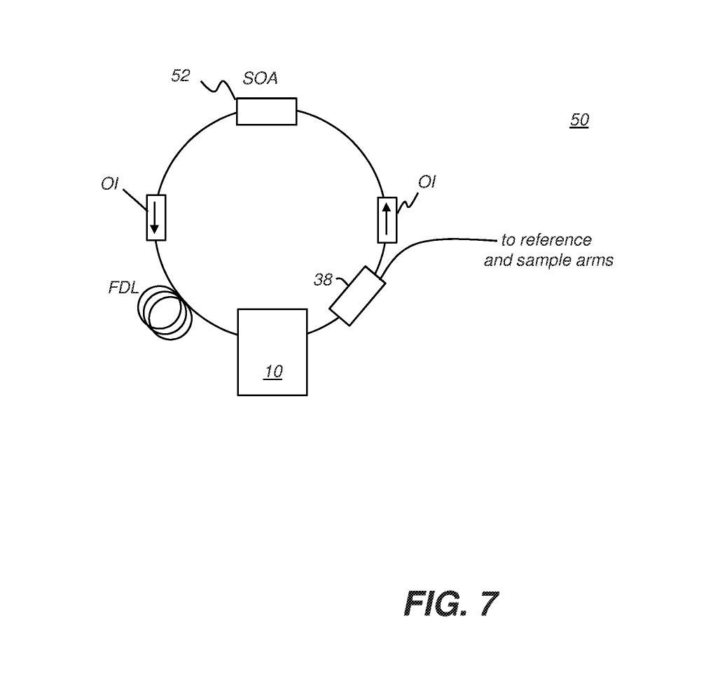

The schematic diagram of FIG. 7 shows components of tuned laser 50 according to an alternate embodiment of the application. Tuned laser 50 is configured as a fiber ring laser having a broadband gain medium such as a semiconductor optical amplifier (SOA) 52. Two optical isolators OI provide protection of the SOA from back-reflected light. A fiber delay line (FDL) determines the effective sweep rate of the laser. Filter 10 has an input fiber and output fiber, used to connect the fiber ring.

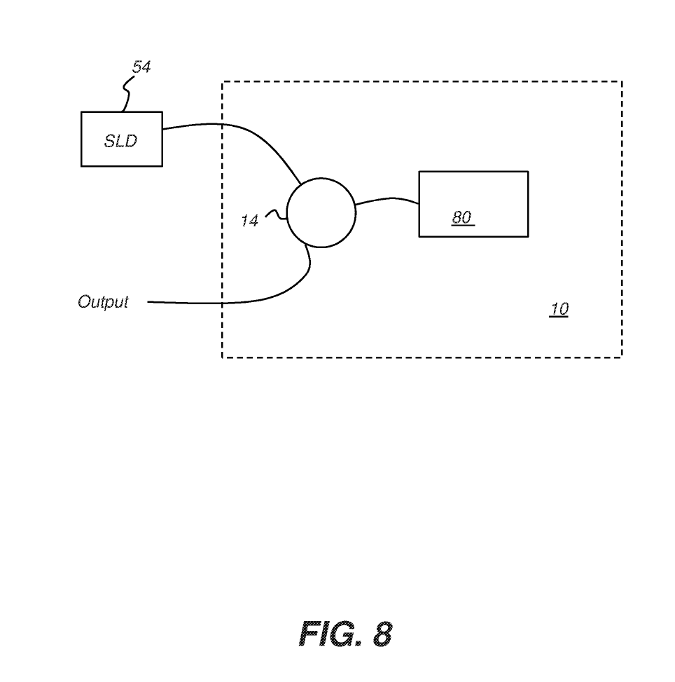

The schematic diagram of FIG. 8 shows the use of programmable filter 10 for selecting a wavelength band from a broadband light source 54, such as a super luminescent diode (SLD). Here, spatial light modulator 80 reflects a component of the broadband light through circulator 14. Circulator 14 is used to direct light to and from the programmable filter 10 along separate optical paths.

### Helical Scan Pattern

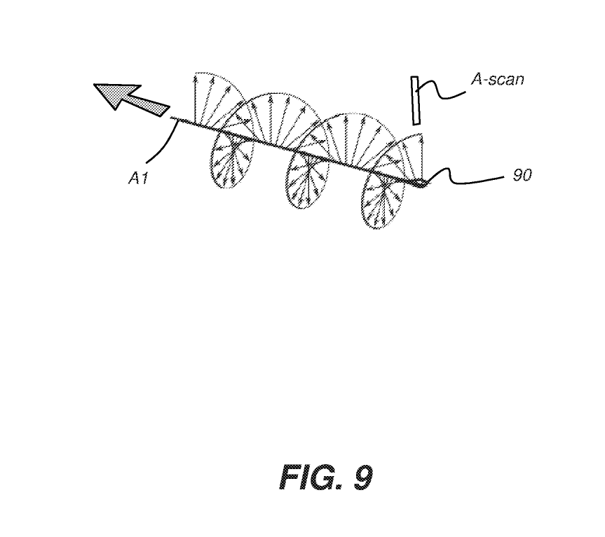

Certain exemplary method and/or apparatus embodiments according to the application are directed to OCT scanning of body cavities, such as the ear canal, respiratory passages, and portions of the throat, for example. Unlike conventional OCT scanning, in which the scan movement for a probe is either manual or in a fixed direction, the scan pattern in exemplary method and/or apparatus embodiments for A-scan acquisition is helical. This scan pattern is advantaged for imaging narrow passages. As FIG. 9 shows, the scanning optics 90 provide illumination and acquire data by rotation with respect to an axis A1 and simultaneous translation along axis A1, acquiring data from the ear canal or other hollow tubular structure. An A-scan is acquired at angular increments, with successive A-scans progressively translated in linear increments along axis A1.

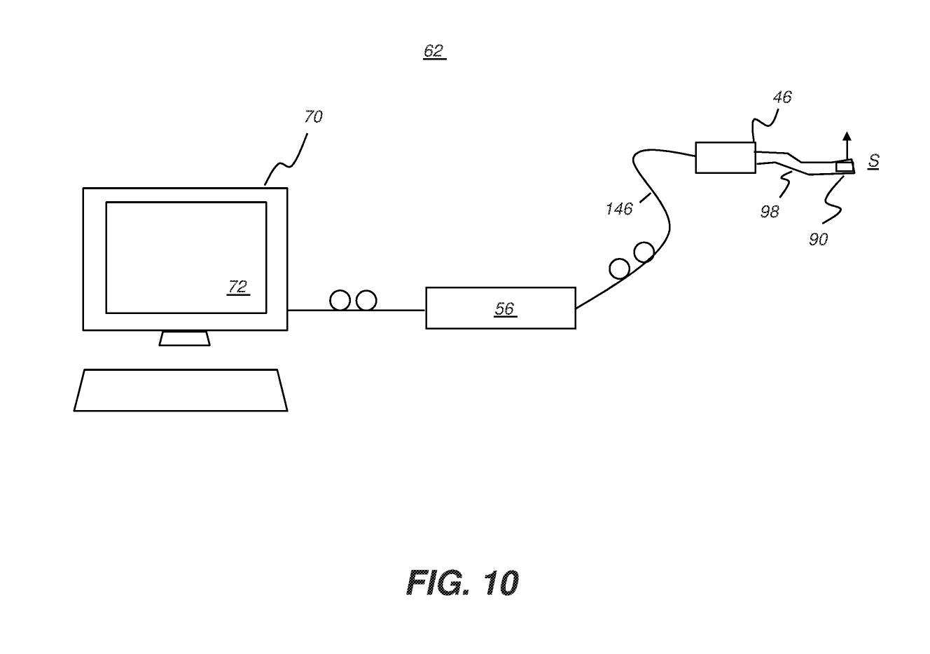

According to an exemplary embodiment of the application, there is provided an apparatus and method for OCT scan acquisition from ear, nose, and throat (ENT) cavities and other body cavities. The schematic diagram of FIG. 10 shows probe 46 and support components particularly suited for forming an OCT imaging system 62 for ENT imaging. An imaging engine 56 includes the light source, fiber coupler, reference arm, spectrometer and OCT detector components described with reference to FIGS. 6A-7. Probe 46, in one exemplary embodiment, connects via an optical fiber 146 and includes the scanner optics 90 or sample arm, but may optionally also contain other elements not provided by imaging engine 56. The probe needle contains micro-scale optics to focus the probe light, including a folding mirror or other optic to redirect the light beam to the imaging site. In order to protect rotating scan components from skin surface contact, the probe needle is preferably housed within a catheter 98. An actuator apparatus drives the probe scanning optics 90 to execute helical scans within catheter 98, with scanning optics 90 simultaneously rotating about an axis while at the same time moving in a direction along the axis, as described with reference to FIG. 9. Catheter 98 can be transparent or have a transparent portion, window or the like for scanning optics 90.

Continuing with FIG. 10, CPU 70, in signal communication with imaging engine 56, includes control logic and display 72. Control software in the CPU 70 initiates and controls operations like optical scanning, volume image reconstruction, distortion and motion correction, digital surface reconstruction, and 3D rendering. Sample light backscattered from sample interferences with reference light. Interference light spectrum is acquired by a spectrometer or balance detector. The data acquisition with the spectrometer is synchronized with the helical scan. Reconstruction using the acquired image content produces a 3D volumetric image from helical scan data.

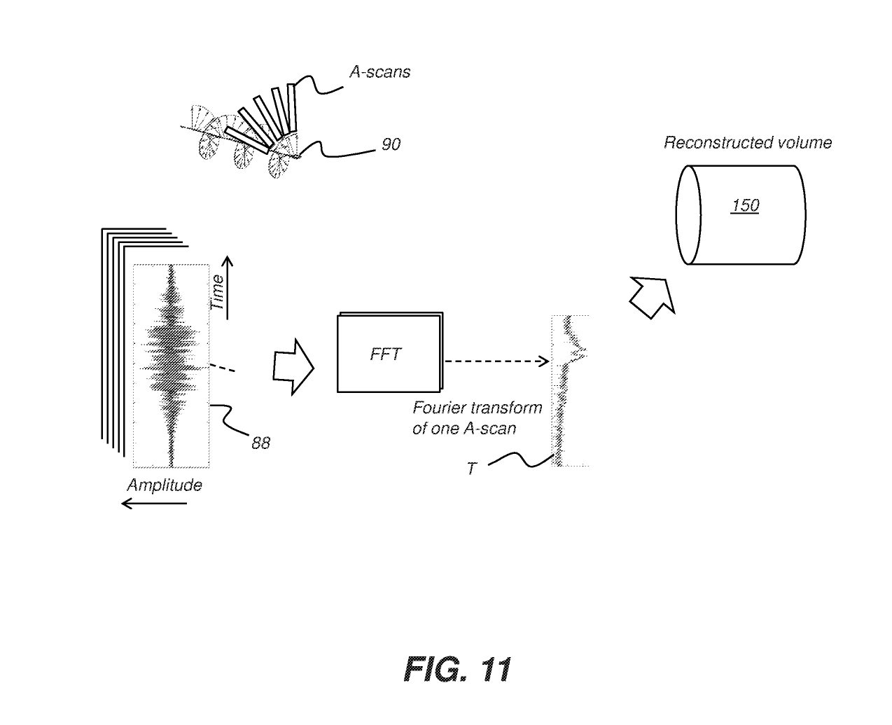

FIG. 11 shows a simplified schematic of portions of an exemplary acquisition and processing sequence for OCT imaging of the ear canal or similar volume using the scan data obtained according to one exemplary embodiment of the application. As shown in FIG. 9, numerous A-scans are obtained, each at a known angular displacement and at a known translational displacement along the scan axis. Each A-scan is processed using an algorithm such as a fast-Fourier transform (FFT) to generate a corresponding transform that encodes the acquired A-scan data. A reconstruction process then generates a reconstructed volume using the transformed A-scan data obtained by helical scanning. It should be noted that the reconstructed volume for the image obtained by probe 46 (FIG. 10) is of the imaged body cavity.

FIG. 11 schematically shows the information acquired during each A-scan. An interference signal 88, shown with DC signal content removed, is acquired over the time interval for each measurement, wherein the signal is a function of the time interval required for the sweep, with the signal that is acquired indicative of the spectral interference fringes generated by combining the light from reference and sample feedback arms of the interferometer (FIGS. 6A, 6B). Fast Fourier transform (FFT) processing generates a transform T for each A-scan. One transform signal corresponding to an A-scan is shown by way of example in FIG. 11. A reconstruction process generates a reconstructed volume 150 from the OCT scan data, applying reconstruction techniques known to those skilled in the art to the cavity scan acquisitions. A portion of the reconstructed volume 150 can be rendered on display 72 (FIG. 10), for example.

In the context of OCT imaging, an “en-face” image is a reconstructed image from an OCT scan that contains a single layer representation of the sample at a given depth. The en-face image need not be planar, however. An en-face image can follow the surface contour of the sample, since each pixel that is used in the en-face image is an equivalent distance from the surface. An image formed using only the pixel on the surface of each scanned point would be a valid en-face image.

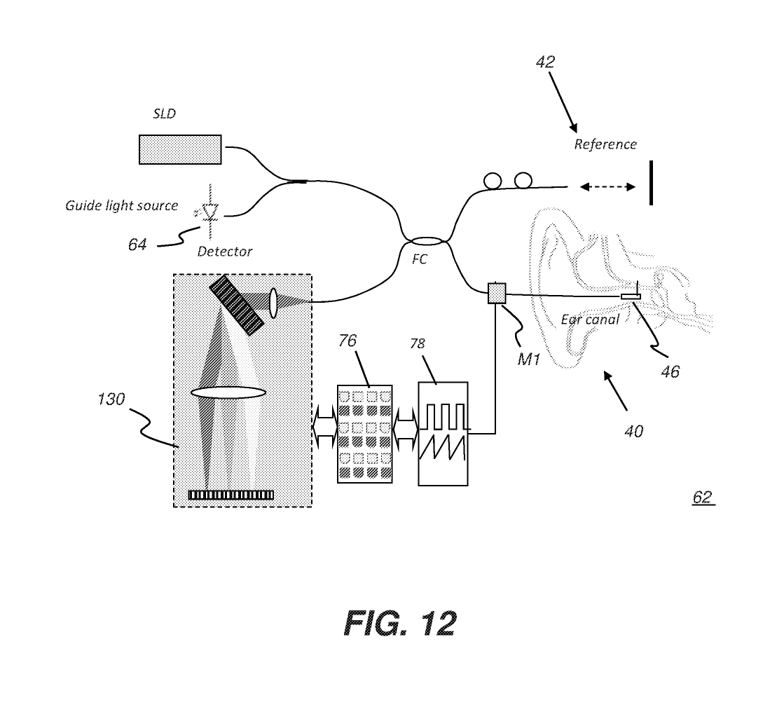

FIG. 12 shows a schematic of imaging system 62 for ear canal characterization. Superluminescent diode SLD is a broadband or low coherence light source. Light is coupled into 50:50 fiber coupler FC. In the reference arm 42, reference light is conveyed over a defined optical path. In sample arm 40, the light beam is conveyed to the ear canal via steering components, optionally assisted by a guide light source 64. Guide light source 64 provides a reference beam of light for helping to guide positioning of imaging system 62 components such as probe 46 and can use a light-emitter of any suitable type. The probe light beam is directed by the scanning optics to be approximately perpendicular to the ear canal wall. An actuator apparatus M1 includes one or more motors that provide actuation that drives the helical scan mechanism in probe 46. Actuator apparatus M1 can provide the simultaneous rotational and translational motion required for helical scanning. Alternately, separate actuators can be used as components of actuator apparatus M1 for rotational and translational motion. One or more of the actuators can be a piezoelectric actuator or leadscrew with stepper motor, for example. Light scattered from the ear canal is coupled back to the probe 46 waveguide and interferes with reference light. The interference light is detected by a spectrometer 130. The helical data scan is synchronized with beam scanning. Reconstruction is performed using each line of interference spectrum data, as described previously. Control electronics 76 control engine operation. A computing unit 78 acquires and processes scan data.

In the exemplary embodiment of FIG. 12, imaging engine design is flexible, and preferably independent from the probe head design. The processing engine can be, for example, a Michelson or Mach-Zender interferometer.

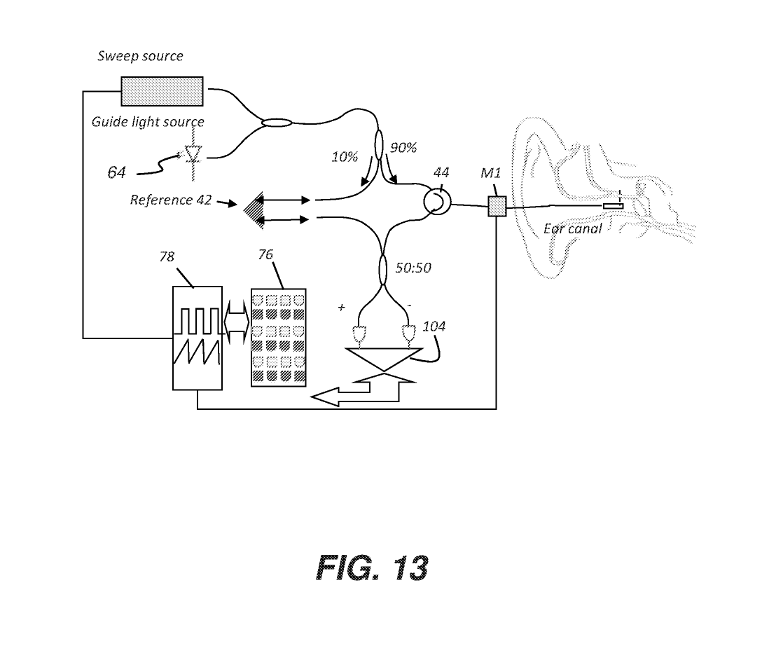

Swept source OCT can be used with an embodiment of imaging system 62, as shown in FIG. 13. In the arrangement shown, circulator 44 directs the sensed signal to a balanced detector 104. Control electronics 76 and computing unit 78 acquire the data from detector 104 and generate image content for display.

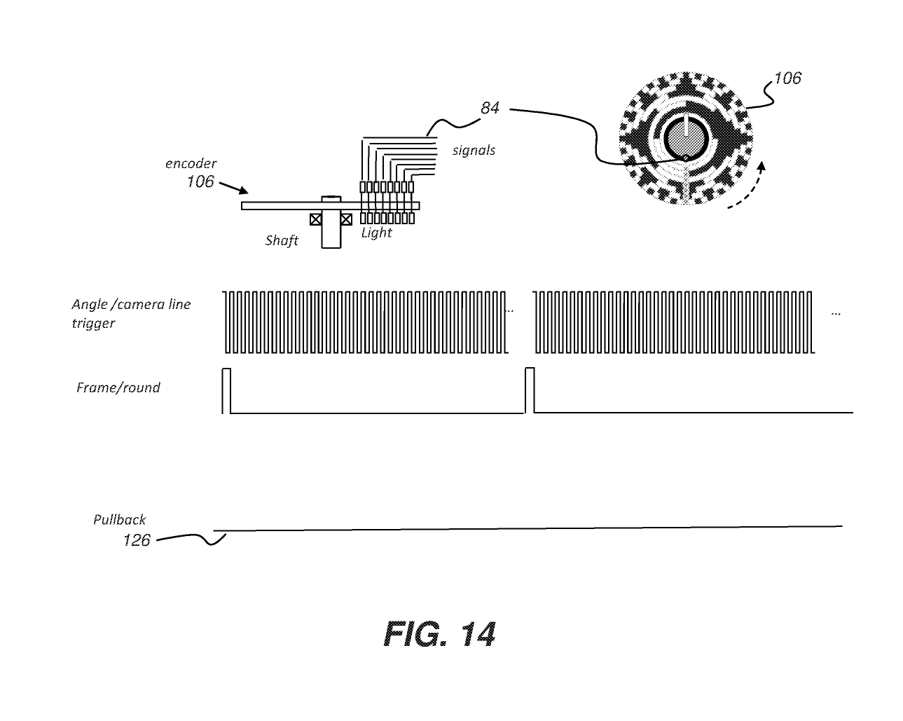

Synchronization of the signal for data acquisition using the helical scan pattern of FIG. 9 can be done in any of several ways. One approach is to use motor rotation of actuator apparatus M1 as the controlling element for acquisition timing. As shown in the timing diagram of FIG. 14, a trigger output signal 84 from an encoder 106 that is coupled to stepping motor of actuator apparatus M1 (FIGS. 12, 13) can be used as the input trigger for spectral data acquisition and for the frequency sweep of the spectral band when using swept source SD-OCT. The data acquisition hardware and the swept source are then actuated by the encoder trigger signal. Data acquisition is timed according to encoder 106 signal transitions, with frames of data sequentially captured in synchronization with each rotation cycle. A continuous pullback signal 126 causes actuator apparatus M1 to translate the probe so that it recedes axially from the ear cavity in synchronization with rotational movement about the same axis.

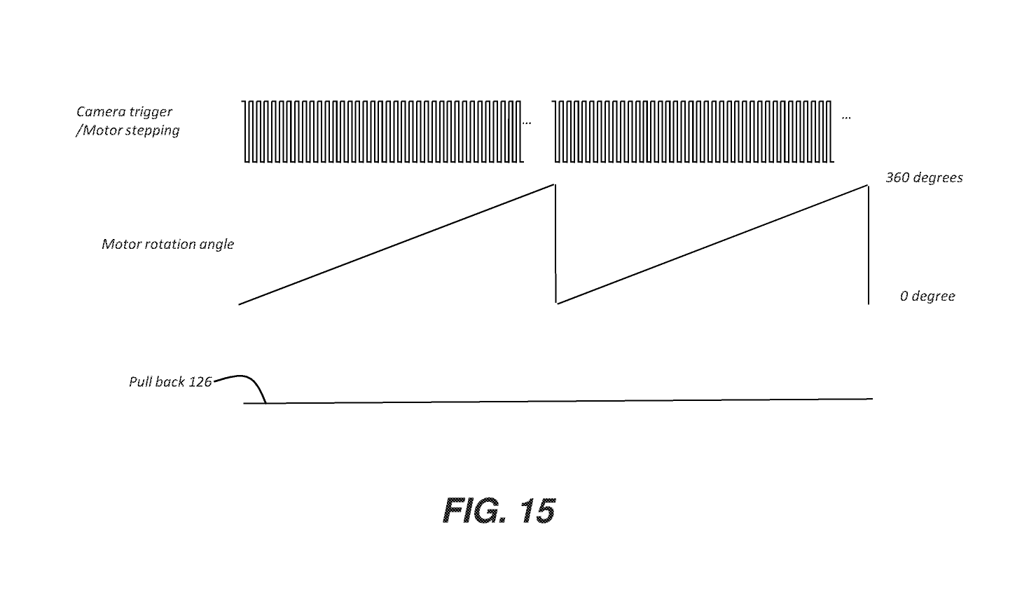

Alternately, trigger signal output from the data acquisition can also be used as a type of master control signal to control motor actuation in actuator apparatus M1. The timing diagram of FIG. 15 shows relative timing and synchronization of a motor in actuator apparatus M1 for changing rotation angle with stepping motor actuation. The trigger signal controls motor rotation. Using the combination of pull back signal 126 and motor step actuation, actuator apparatus M1 generates the helical scan pattern described with reference to FIG. 9. Image acquisition and motor stepping can share the same clock signal.

If swept source SD-OCT is used, the trigger signal can be obtained from the swept source itself. As another option, a series of control logic signals that drive the individual components can be used for synchronization of image acquisition and scan movement by actuator apparatus M1.

### Design of the ENT Probe

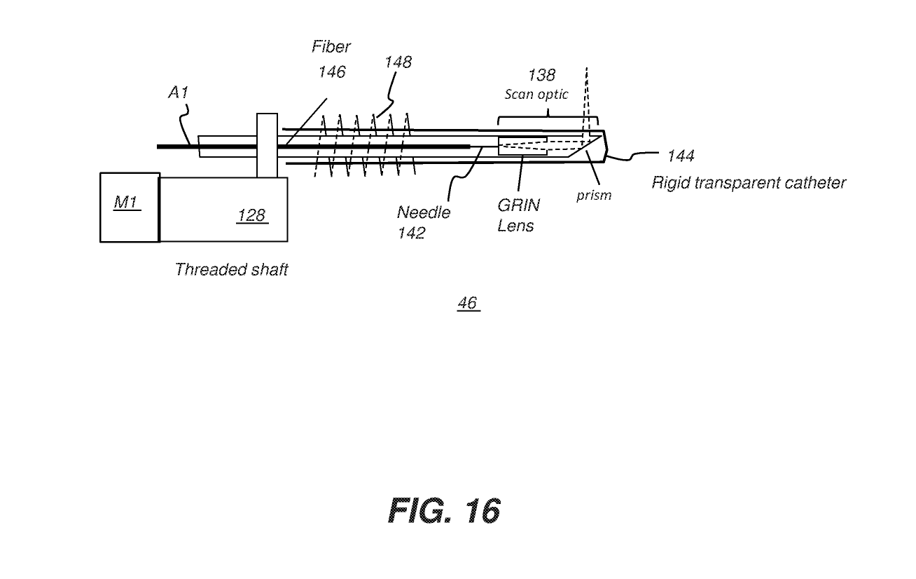

ENT probe 46 can have any of a number of arrangements suitable for the imaging task. As shown in the diagram of FIG. 16, probe 46 has a thin needle 142, which can be a flexible wire or other material, for example, provided at the end of an optical fiber 146, housed within a catheter 144, having a transparent outer housing. Needle 142 defines axis of rotation A1 which also defines the path for linear translation by which actuator apparatus M1 enables the helical scan pattern described previously with respect to FIG. 9.

ENT probe 46 can have a catheter 144 that allows insertion into the ear canal, nose cavity or throat, while protecting and stabilizing the scanner components. Probe 46 can be controlled to become rigid with externally applied force so that the probe remains static, holding a fixed shape during imaging. Probe 46 can be fairly rigid and needle-like because the ear canal is relatively unobstructed and straight. Helical scan 148 is used for the optical scanning components, as described previously. To provide the helical scan pattern, a threaded shaft 128 driven by actuator apparatus M1 retracts the probe 46 linearly or axially from its original position during rotation of scanning optics 138, such as a mirror or prism. The helical movement that is effected is thus able to move the scanning optics 138 to scan the balance of the ear canal with catheter 144 in place. Encasement within catheter 144 allows the helical scan movement to take place while probe 46 is in a stationary position.

The probe configuration can be modified as needed, depending on the particular imaging application. Different probes could be used for scanning different body cavities or for scanning cavities of different widths, such as for adult or child patients. Different probes could also be provided with different helical scan pitch, allowing variable resolution according to probe type. Switching the probe for an alternate configuration simply requires connecting a different probe to the fiber optic connection and actuator apparatus control signals, using the power and signal cabling provided.

An optic such as a gradient-index (GRIN) lens as part of scanning optics 138 can provide suitable focusing during scanning of light within the probe 46. The small size of the GRIN lens is helpful for providing compact optics suited to the tight dimensioning needed. However, other optics capable of providing similar functionality can be used.

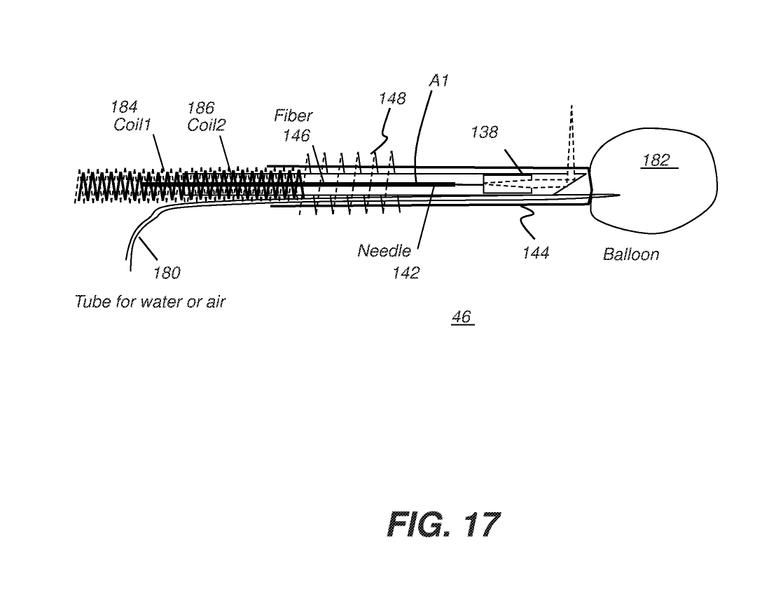

The diagram of FIG. 17 shows a probe 46 arrangement that is configured for stabilizing position within the ear canal or other body cavity. Catheter 144 is flexible for insertion and initial positioning, but can be stiffened or made rigid using tension applied to a tensioning coil 184. Tensioning coil 184 can thus be configured for variable stiffness under external force when the probe 46 has been positioned at the correct imaging site. A second rotating inner coil 186 transmits the helical scan pattern to the needle 142 for scan execution. To stabilize probe 46 position, a balloon or other type of inflatable bladder 182 extending from the tip of catheter 144 can be inflated, such as with air or water, through a tube 180. The balloon or bladder 182 provides an adjustable gripping surface that presses against adjacent sites within the ear canal or other anatomical cavity to further fix the catheter 144 in position.

Distortion correction and motion calibration techniques familiar to those skilled in the image acquisition and motion control arts can be used to help coordinate helical scan movement and to generate the correct shape of the imaged 3D volume from the scan data that is obtained.

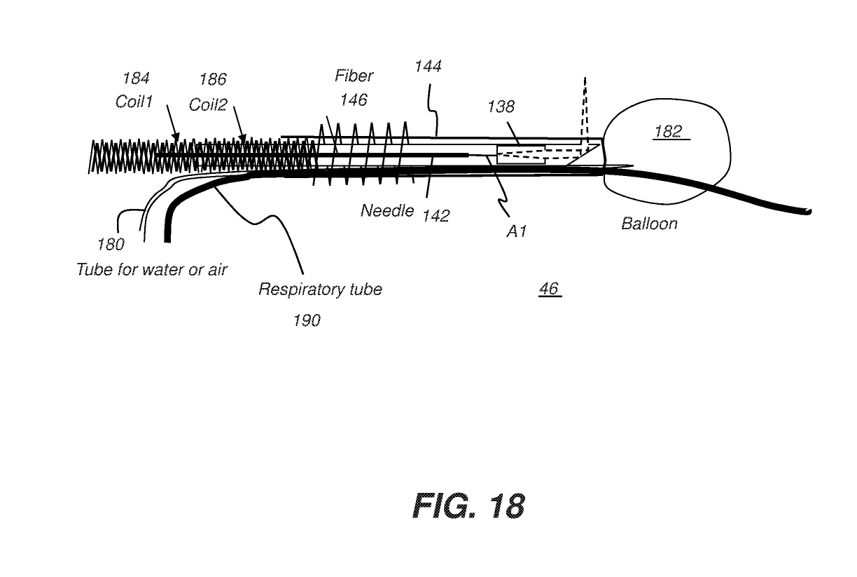

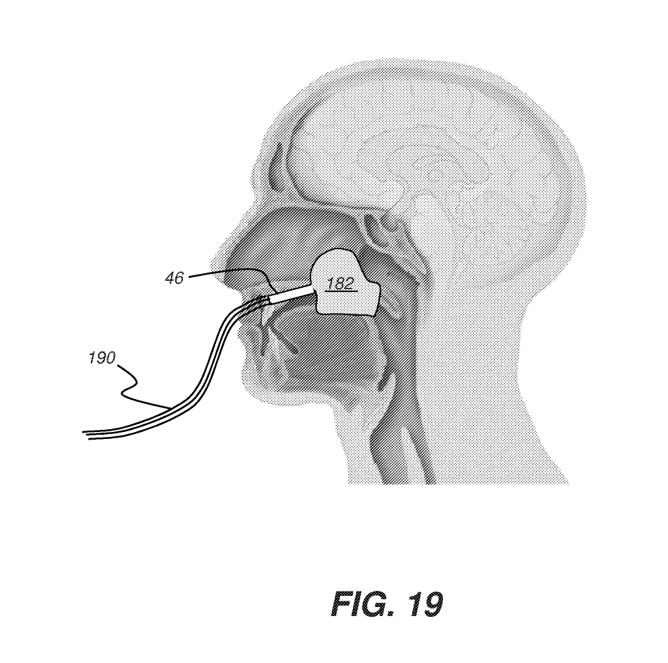

The schematic diagram of FIG. 18 and cross-sectional diagram of FIG. 19 show a probe 46 configured for stabilization as shown in FIG. 17 with addition of an auxiliary respiratory tube 190 for providing air or oxygen to the patient. Respiratory tube 190 can be provided within or alongside catheter 144.

Consistent with exemplary embodiments herein, a computer program can use stored instructions that perform on image data that is accessed from an electronic memory. As can be appreciated by those skilled in the image processing arts, a computer program for operating the imaging system and probe and acquiring image data in exemplary embodiments of the application can be utilized by a suitable, general-purpose computer system operating as CPU 70 as described herein, such as a personal computer or workstation. However, many other types of computer systems can be used to execute the computer program of the present invention, including an arrangement of networked processors, for example. The computer program for performing exemplary method embodiments may be stored in a computer readable storage medium. This medium may include, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. Computer programs for performing exemplary method embodiments may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the application, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer is also considered to be a type of memory, as the term is used in the application. Memory is also used as the data workspace for executing and storing intermediate and final results of calculations and other processing. Computer-accessible memory can be volatile, non-volatile, or a hybrid combination of volatile and non-volatile types.

It will be understood that computer program products of the application may make use of various image manipulation algorithms and processes that are well known. It will be further understood that computer program product exemplary embodiments of the application may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product exemplary embodiments of the application, are not specifically shown or described herein and may be selected from such algorithms, systems, hardware, components and elements known in the art.

Certain exemplary method and/or apparatus embodiments according to the application can provide helical OCT scan acquisition from ENT cavities. In one exemplary embodiment, probes for helical OCT scan acquisition are switchable coupled to an imaging system for respectively imaging each of the ear, nose, and throat body cavities. Exemplary embodiments according to the application can include various features described herein (individually or in combination).

While the invention has been illustrated with respect to one or more implementations, alterations and/or modifications can be made to the illustrated examples without departing from the spirit and scope of the appended claims. In addition, while a particular feature of the invention can have been disclosed with respect to only one of several implementations/embodiments, such feature can be combined with one or more other features of the other implementations/embodiments as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. Other embodiments of the invention will be apparent to those skilled in the art from consideration of the specification and practice of the invention disclosed herein. It is intended that the specification and examples be considered as exemplary only, with a true scope and spirit of the invention being indicated by at least the following claims.

1. An apparatus for imaging a body cavity comprising:
a) a light source that generates short coherence light;
b) a probe housed within a catheter, wherein the probe has a rotatable scanner optic that is disposed to convey the coherent light outwards from the light source to the body cavity and to obtain scattered light from the body cavity;
c) an actuator apparatus coupled to the probe and configured to perform a helical scan by simultaneously rotating the scanner optic about an axis within the catheter and translating the scanner optic along the axis;
d) a signal detector that obtains an interference signal between a first portion of the coherent light scattered from the body cavity and a second portion of the coherent light reflected from a reference; and
e) a processor that is programmed with instructions that coordinate helical scan timing of the scanner optic and acquisition of data from the signal detector and further programmed with instructions for volume image reconstruction.

2. The apparatus of claim 1 wherein the light source is a superluminescent light emitting diode or a swept frequency light source.

3. The apparatus of claim 1 further comprising a fiber that conducts the coherent light to a gradient-index lens.

4. The apparatus of claim 1 further comprising a stabilizing inflatable bladder coupled to the probe.

5. The apparatus of claim 1 wherein the generated light is of wavelengths above a threshold wavelength, wherein the light source is emissive over a wavelength band, and wherein the wavelength band is at least 20 nm.

6. The apparatus of claim 1 wherein the actuator apparatus comprises a piezoelectric actuator or a leadscrew with a stepper motor.

7. The apparatus of claim 1 wherein the scanner optic comprises a gradient-index lens, a prism, or a mirror.

8. The apparatus of claim 1 wherein the body cavity is in the ear, nose, or throat.

9. The apparatus of claim 1 further comprising a respiratory tube inside or along the catheter.

10. The apparatus of claim 1 wherein the probe further comprises a tensioning coil, and wherein the probe further comprises a guide light.

11. The apparatus of claim 1 wherein the processor is further programmed with instructions for display, storage, or transfer of images from the acquired data.

12. The apparatus of claim 1 wherein the probe is switchable between two or more of ear, nose, and throat body cavities.

13. A method for imaging a body cavity comprising:
a) generating a low coherence light;
b) providing a probe housed within a transparent catheter, wherein the probe has a rotatable scanner optic that is disposed to convey the light from the light source to and from the body cavity;
c) rotating the scanner optic about an axis within the catheter and simultaneously translating the scanner optic along the axis to provide a helical scan;
d) obtaining an interference signal between a first portion of the coherent light scattered from the body cavity and a second portion of the coherent light reflected from a reference; and
e) processing the obtained interference signal to reconstruct a volume image of the body cavity.

14. The method of claim 13 further comprising rendering a portion of the volume image to a display, wherein the body cavity is in the ear, nose, or throat, and wherein the probe is switchable between two or more of ear, nose, and throat body cavities.

15. The method of claim 13 further comprising:
stiffening the catheter in position within the body cavity; and
stabilizing the catheter position by inflating at least one bladder extending from the catheter.
